(19) United States
(12) Patent Application Publication
(10) Pub. No.: US 2002/0177231 A1
(43) Pub. Date: Nov. 28, 2002

(54) LEUPAXIN MATERIALS AND METHODS

(76) Inventors: DONALD E. STAUNTON, KIRKLAND, WA (US); BRIAN P. LIPSKY, SEATTLE, WA (US); PATRICK W. GRAY, SEATTLE, WA (US)

Correspondence Address: GRETA E NOLAND, MARSHALL O"TOOLE GERSTEIN MURRAY&BORUN, 6300 SEARS TOWER, 233 SOUTH WACKER DRIVE, CHICAGO, IL, 606066402

(21) Appl. No.: 09/211,424

(22) Filed: Dec. 15, 1998

(51) Int. Cl. G01N 31/00; C07K 17/00; C07K 14/00

(52) U.S. Cl. 436/6; 530/350

(57) ABSTRACT

Disclosed are novel leupaxin polypeptides, polynucleotides encoding the polypeptides, expression constructs comprising the polynucleotides, host cell transformed with the polynucleotides, methods to produce the polypeptides, antibodies and binding partners specific for the polypeptides, methods to identify modulators of the polypeptides, and methods to identify modulators of polypeptide expression.

### BACKGROUND OF THE INVENTION

Cell adhesion, spreading, and migration are mediated by integrin interactions with extracellular and cell surface ligands [Gumbiner, Cell, 84:345-357 (1996); Hynes, et al., Cell, 68:303-322 (1992)]. In adherent cell types such as epithelial cells and fibroblasts, readily identifiable complexes of cytoplasmic proteins localize at sites of integrin-dependent close cell contact with substratum. These complexes are designated focal adhesions/contacts and have been implicated in the regulation of cell locomotion, survival and proliferation [Lee, et al., Trends Cell Biol., 3:366-370 (1993); Ruoslahti, et al., Cell, 77:477-478 (1994)].

Focal adhesions are rich in tyrosine phosphorylated proteins which suggests a role for tyrosine kinases in integrin signaling [Parsons, et al., Curr. Opin. Cell. Biol. 9:187-192 (1997); Miyamoto, et al., J. Cell. Biol. 131:791-805 (1995)]. Protein tyrosine kinases that are found in focal adhesions includes focal adhesion kinase (FAK), src, and src-family kinases. A FAK-related protein, PYK2, has also been identified and designated by various groups as CAKβ [Sasaki, et al., J. Biol. Chem. 270:21206-21219 (1995)], RAFK [Avraham, et al., J. Biol. Chem. 270:27742-27751 (1995)] and CADTK [Yu, et al., J. Biol. Chem., 77:29993-29998 (1996)]. PYK2 and FAK are closely related in overall structure and both are phosphorylated on tyrosine in response to integrin engagement, T cell receptor engagement, or chemokine stimulation. These stimuli all modulate integrin dependent adhesion. PYK2 and FAK both associate with paxillin, p130cas, and src [Ganju, et al., J. Exp. Med., 185:1055-1063 (1997); Astier, et al., J. Biol. Chem., 272:228-232 (1997)]. Although PYK2 possesses a focal adhesion targeting domain that is highly homologous to the corresponding region in FAK, PYK2 displays a more diffuse cytoplasmic distribution than FAK, with only a small percentage of the protein found in focal adhesions [Matsuya, et al., J. Biol. Chem., 273:1003-1014 (1998)]. These observations suggest that FAK and PYK2 have both overlapping and distinct functions.

In addition to FAK and paxillim, tyrosine phosphoproteins present in focal adhesions include vinculin, zyxin, and the paxillin-like protein Hic-5 [Matsuya, et al., J. Biol. Chem., 273:1003-1014 (1998)]. Phosphorylation of tyrosine on these proteins can regulate interaction with proteins that possess src homology 2 domains (S H2) and phosphotyrosine binding (PTB) domains suggesting that tyrosine kinase activity plays a role in protein-protein interactions in these dynamic focal adhesion complexes [Miyamoto, et al., J. Cell. Biol. 131:791-805 (1995) Van der Gear, et al., Trends Biochem. Sci., 20:277-280 (1995)].

In addition, several focal adhesion proteins possess LIM domains that can mediate interactions with other proteins [Schmeichel, et al., Cell, 79:211-219 (1994)]. LIM domains are approximately 50 residues in length and contain conserved cysteine, histidine, and aspartate residues that form zinc binding modules [Perez-Alvarado, et al., Nat. Struct. Biol. 1:388-398 (1994); Kosa, et al., Biochemistry, 33:468-477 (1994); Michelsen, et al., J. Biol. Chem. 269:11108-11113. (1994)]. Paxillin, Hic-5, zyxin, and cysteine-rich protein (CRP) contain a tandem array of three or four LIM domains in the carboxyl-terminal regions. Individual LIM domains demonstrate specificity for binding different proteins or protein motifs. For example, the zyxin LIM1 domain supports a binding interaction with CRP [Schmeichel and Beckerle, Cell 79:211-219 (1994)] and paxillin LIM3 has been shown to participate in localization of paxillin to focal adhesions [Brown, et al., J. Cell. Biol. 135:1109-1123 (1996)]. LIM domains in Enigma, a protein that interacts with the insulin receptor and the receptor tyrosine kinase Ret, bind to specific tyrosine-containing tight-turn motifs [Wu, et al., J. Biol. Chem. 271:15934-15941 (1996)].

Other short sequences designated leucine-aspartate (LD) motifs in the amino terminal region of paxillin participate in the binding to FAK and to vinculin [Brown, et al., J. Cell. Biol. 135:1109-1123 (1996)]. Domains of this type invariably include a signature leucine-aspartate dipeptide sequence at the amino terminus and were first characterized in paxillin as thirteen amino acid motifs that participate in specific protein binding. Paxillin regions LD2 and LD3 have been implicated in binding to FAK to localize the protein at focal adhesions, and domain LD2 is believed to mediate paxillin binding to vinculin. FAK and vinculin regions designated PBS participate in binding with paxillin LD motifs [Tachibana, J. Exp. Med. 182:1089-1099 (1995)]. In paxillin, the LD regions appear to participate in localization of FAK at focal adhesions. Thus focal adhesion proteins such as paxillin contain multiple binding domains and likely serve as scaffolds to localize and regulate specific effector molecules to a subcellular site.

Thus there exists a need in the art to identify proteins which mediate integrin binding and, in turn, modulate cell adhesion, spreading, and migration.

### BRIEF SUMMARY OF THE INVENTION

In brief, the present invention provides polypeptides and underlying polynucleotides for a novel family of proteins designated leupaxins. The invention includes both naturally occurring and non-naturally occurring leupaxin polynucleotides and polypeptide products thereof. Naturally occurring leupaxin products include distinct gene and polypeptide species within the leupaxin family; these species include those which are expressed within cells of the same animal as well as corresponding species homologs expressed in cells of other animals. Within each leupaxin species, the invention further provides splice variants encoded by the same polynucleotide but which arise from distinct mRNA transcripts. Non-naturally occurring leupaxin products include variants of the naturally occurring products such as analogs (i.e., wherein one or more amino acids are added, substituted, or deleted) and those leupaxin products which include covalent modifications (i.e., fusion proteins, glycosylation variants, Met⁻¹-leupaxin, Met⁻²-Lys⁻¹-leupaxin, Gly⁻¹-leupaxin and the like). The leupaxin family of proteins is distinguished from previously known localization families of proteins in that leupaxins include distinct amino acid sequences which suggest interaction with unique ligands as well as distinct modes of regulation. In a preferred embodiment, the invention provides a polynucleotide comprising the sequence set forth in SEQ ID NO: 1. The invention also embraces polynucleotides encoding the amino acid sequence set out in SEQ ID NO: 2. A presently preferred polypeptide of the invention comprises the amino acid sequence set out in SEQ ID NO: 2.

The present invention provides novel purified and isolated polynucleotides (e.g., double stranded and single stranded DNA sequences and RNA transcripts, both sense and complementary antisense strands, including splice variants thereof) encoding the human leupaxins. DNA sequences of the invention include genomic and cDNA sequences (double stranded and single stranded sequences) as well as wholly or partially chemically synthesized DNA sequences. “Chemically synthesized,” as used herein and is understood in the art, refers to polynucleotides produced by purely chemical, as opposed to enzymatic, techniques. “Wholly” synthesized DNA sequences are therefore produced entirely by chemical means, and “partially” synthesized DNAs embrace those wherein only portions of the resulting DNA were produced by chemical means. A preferred DNA sequence encoding a human leupaxin polypeptide is set out in SEQ ID NO: 1. The worker of skill in the art will readily appreciate that the preferred DNA of the invention comprises a double stranded molecule, for example the molecule having the sequence set forth in SEQ ID NO: 1 along with the complementary molecule (the “non-coding strand” or “complement”) having a sequence deducible from the sequence of SEQ ID NO: 1 according to Watson-Crick base paring rules for DNA. Also preferred are polynucleotides encoding the leupaxin polypeptide of SEQ ID NO: 2. The invention further embraces species, preferably mammalian, homologs of the preferred human leupaxin DNA.

The invention also embraces DNA sequences encoding leupaxin species which hybridize under moderately stringent conditions to the complete non-coding strand (complement) or distinct regions thereof, of the polynucleotide in SEQ ID NO: 1. DNA sequences encoding leupaxin polypeptides which would hybridize thereto but for the redundancy of the genetic code are contemplated by the invention. Exemplary moderate hybridization conditions are as follows: hybridization at 60° C. in 5×SSC, and washing at 60° C. in 1×SSC. It is understood in the art that conditions of equivalent stringency can be achieved through variation of temperature and buffer, or salt concentration as described in Ausebel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

Autonomously replicating recombinant expression constructions such as plasmid and viral DNA vectors incorporating leupaxin sequences are also provided. Expression constructs wherein leupaxin-encoding polynucleotides are operatively linked to an endogenous or exogenous expression control DNA sequence and a transcription terminator are also provided.

According to another aspect of the invention, host cells are provided, including prokaryotic and eukaryotic cells, either stably or transiently transformed or transfected with DNA sequences of the invention in a manner and under conditions which permits expression of leupaxin polypeptides of the invention. Host cells of the invention are a valuable source of immunogen for development of antibodies specifically immunoreactive with leupaxin. Host cells of the invention are also conspicuously useful in methods for large scale production of leupaxin polypeptides wherein the cells are grown in a suitable culture medium and the desired polypeptide products are isolated from the cells or from the medium in which the cells are grown by, for example, immunoaffinity purification, gel permeation chromatography, ion exchange chromatography, gel electrophoresis, Western blotting, immunoprecipitation, or any of a number of other purification techniques well known and routinely practiced in the art. Purification techniques for isolating leupaxin polypeptides of the invention can be employed alone or in combination.

Knowledge of leupaxin DNA sequences allows for modification of cells to modulate, increase or decrease, expression of endogenous leupaxin in host cell which naturally include polynucleotides that encode leupaxin. Cells can be modified (e.g., by homologous recombination) to provide modified leupaxin expression by replacing, in whole or in part, the naturally occurring leupaxin promoter with all or part of a heterologous promoter so that the cellular expression of leupaxin occurs at higher or lower levels. The heterologous promoter is inserted in such a manner that it is operatively linked to leupaxin encoding sequences. See, for example, PCT International Publication No. WO 94/12650, PCT International Publication No. WO 92/20808, and PCT International Publication No. WO91/09955. The invention also contemplates that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the leupaxin coding sequence, amplification of the marker DNA by standard selection methods results in co-amplification of the expression product of the leupaxin coding sequences in the cells.

The DNA sequence information provided by the present invention also makes possible the development, through e.g., homologous recombination or “knock-out” strategies [Capecchi, Science 244:1288-1292 (1989)], of animals that fail to express functional leupaxin or that express a variant of leupaxin. Such animals are useful as models for studying the in vivo activities of leupaxin and modulators of leupaxin.

The invention also provides purified and isolated mammalian leupaxin polypeptides. Presently preferred is a leupaxin polypeptide comprising the amino acid sequence set out in SEQ ID NO: 2. Leupaxin polypeptides of the invention may be isolated from natural cell sources or may be chemically synthesized, but are preferably produced by recombinant procedures involving host cells of the invention. Use of mammalian host cells is expected to provide for such post-translational modifications (e.g., glycosylation, truncation, lipidation, ubiquitination, and phosphorylation) as may be needed to confer optimal biological activity on recombinant expression products of the invention. Leupaxin products of the invention may be full length polypeptides, biologically active fragments, or variants thereof which retain specific leupaxin biological or immunological activity. Variants may comprise leupaxin polypeptide analogs wherein one or more of the specified (i.e., naturally encoded) amino acids is deleted or replaced or wherein one or more non-specified amino acids are added: (1) without loss of one or more of the biological activities or immunological characteristics specific for leupaxin; or (2) with specific disablement of a particular biological activity of leupaxin. Leupaxin polypeptide fragments of the invention include specific protein binding domains including regions that participate in cytoplasmic localization. Presently preferred polypeptide fragments include regions comprising LD and LIM domains of the polypeptide set out in SEQ ID NO:2. LD domain fragments of leupaxin are exemplified by polypeptides comprising amino acid residues 4 through 15, 40 through 51, 93 through 104, and 128 through 139 as set out in SEQ ID NO: 2, as well as corresponding LD regions in other leupaxin polypeptides embraced by the invention. LIM domain fragments of leupaxin are exemplified by polypeptides comprising amino acid residues 152 through 202, 211 through 261, 270 through 320, and 329 through 379 as set out in SEQ ID NO: 2, as well as corresponding LIM regions in other leupaxin polypeptides embraced by the invention.

Variant products of the invention include mature leupaxin products as well as leupaxin products including additional amino terminal residues. Leupaxin products having an additional methionine residue at position -1 (Met⁻¹-leupaxin) are contemplated, as are leupaxin products having additional methionine and lysine residues at positions -2 and -1 (Met⁻²-Lys⁻¹-eupaxin). Also contemplated are leupaxin products having multiple Met-Lys additional residues, in addition to other additional sequences which permit enhanced expression and/or recovery of leupaxin products of the invention. Variants of these types are particularly useful for recombinant protein production in bacterial cell types.

The invention also embraces leupaxin variants having additional amino acid residues which result from use of specific expression systems. For example, use of commercially available vectors that express a desired polypeptide such as a glutathione-S-transferase (GST) fusion product provide the desired polypeptide having an additional glycine residue at position -1 as a result of cleavage of the GST component from the desired polypeptide. Variants which result from expression in other vector systems are also contemplated.

The invention further embraces leupaxin products modified to include one or more water soluble polymer attachments. Particularly preferred are leupaxin products covalently modified with polyethylene glycol (PEG) subunits. Water soluble polymers may be bonded at specific positions, for example at the amino terminus of the leupaxin products, or randomly attached to one or more side chains of the polypeptide.

Also comprehended by the present invention are antibodies (e.g., monoclonal and polyclonal antibodies, single chain antibodies, chimeric antibodies, CDR-grafted antibodies, humanized antibodies, and the like) and other binding proteins specific for leupaxin products or fragments thereof The term “specific for” indicates that the variable regions of the antibodies of the invention recognize and bind leupaxin polypeptides exclusively (i.e., able to distinguish specific leupaxin polypeptides from the family of leupaxin polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 14. Antibodies that recognize and bind fragments of the leupaxin polypeptides of the invention are also contemplated, provided that the antibodies are first and foremost specific for, as defined above, leupaxin polypeptides. As with antibodies that are specific for full length leupaxin polypeptides, antibodies of the invention that specifically recognize leupaxin fragments are those which can distinguish specific leupaxin polypeptides from the family of leupaxin polypeptides despite inherent sequence identity, homology, or similarity found in the family of proteins.

Specific binding proteins can be developed using isolated or recombinant leupaxin products, leupaxin variants, or cells expressing a modified leupaxin product such that the antigenic leupaxin product is expressed on the cells surface. Binding proteins are useful for purifying leupaxin products and detection or quantification of leupaxin products in fluid and tissue samples using known immunological procedures. Binding proteins are also manifestly useful in modulating (i.e., blocking, inhibiting, or stimulating) biological activities of leupaxin, especially those activities involved in signal transduction. Anti-idiotypic antibodies specific for anti-leupaxin antibodies are also contemplated.

The scientific value of the information contributed through the disclosures of DNA and amino acid sequences of the present invention is manifest. As one series of examples, knowledge of the sequence of a cDNA for leupaxin makes possible through use of Southern hybridization or polymerase chain reaction (PCR) the identification of genomic DNA sequences encoding leupaxin and leupaxin expression control regulatory sequences such as promoters, operators, enhancers, repressors, and the like. DNA/DNA hybridization procedures carried out with DNA sequences of the invention under moderately stringent conditions are likewise expected to allow the isolation of DNAs encoding allelic variants of leupaxin; allelic variants are known in the art to include structurally related proteins sharing one or more of the biochemical and/or immunological properties specific to leupaxin. Similarly, non-human species genes encoding proteins homologous to leupaxin can also be identified by Southern and/or PCR analysis and are useful in animal models for leupaxin-related disorders. As an alternative, complementation studies can be useful for identifying other human leupaxin products as well as non-human proteins, and DNAs encoding the proteins, sharing one or more biological properties of leupaxin.

Polynucleotides of the invention are also useful in hybridization assays to detect the capacity of cells to express leupaxin. Polynucleotides of the invention may also be the basis for diagnostic methods useful for identifying a genetic alteration(s) in a leupaxin locus that underlies a disease state or states.

Also made available by the invention are anti-sense polynucleotides which hybridize to polynucleotides encoding leupaxin. Full length and fragment antisense polynucleotides are provided. The worker of ordinary skill will appreciate that fragment antisense molecules of the invention include (i) those which specifically recognize and hybridize to leupaxin DNA (as determined by sequence comparison of DNAs encoding leupaxin to DNA encoding other known molecules) as well as (ii) those which recognize and hybridize to DNA encoding other members of the leupaxin family of proteins. Antisense polynucleotides that hybridize to multiple DNA encoding other members of the leupaxin family of proteins are also identifiable through sequence comparison to identify characteristic, or signature, sequences for the leupaxin family of molecules. Antisense polynucleotides are particularly relevant to regulating expression of leupaxin by those cells expressing leupaxin mRNA.

The DNA and amino acid sequence information provided by the present invention also makes possible the systematic analysis of the structure and function of leupaxins. DNA and amino acid sequence information for leupaxin also permits identification of binding partner compounds with which a leupaxin polypeptide or polynucleotide will interact. Agents that modulate (i.e., increase, decrease, or block) leupaxin activity or expression may be identified by incubating a putative modulator with a leupaxin polypeptide or polynucleotide and determining the effect of the putative modulator on leupaxin activity or expression. The selectivity of a compound that modulates the activity of the leupaxin can be evaluated by comparing its binding and/or modulating activity on leupaxin to its binding and/or modulating activity on other proteins. Cell based methods, such as di-hybrid assays to identify DNAs encoding binding compounds and split hybrid or reverse di-hybrid assays to identify inhibitors of leupaxin polypeptide interaction with a known binding polypeptide, as well as in vitro methods to identify both known and heretofore unknown binding ligands, including assays wherein a leupaxin polypeptide, leupaxin polynucleotide, or a binding partner thereof is immobilized, and solution assays are contemplated by the invention.

Selective modulators may include, for example, antibodies and other proteins or peptides which specifically bind to a leupaxin polypeptide or a leupaxin-encoding nucleic acid, oligonucleotides which specifically bind to a leupaxin polypeptide or a leupaxin-encoding gene sequence, and other non-peptide compounds (e.g., isolated or synthetic organic and inorganic molecules) which specifically interact with a leupaxin polypeptide or underlying nucleic acid. Mutant leupaxin polypeptides which affect the enzymatic activity or cellular localization of the wild-type leupaxin polypeptides are also contemplated by the invention. Presently preferred targets for the development of selective modulators include, for example: (1) regions of the leupaxin polypeptide which contact other proteins and/or localize the leupaxin polypeptide within a cell, (2) regions of the leupaxin polypeptide which bind substrate, (3) allosteric regulatory binding sites of the leupaxin polypeptide, (4) phosphorylation site(s) of the leupaxin polypeptide and (5) regions of the leupaxin polypeptide which are involved in multimerization of leupaxin subunits. Still other selective modulators include those that recognize specific leupaxin-encoding and regulatory polynucleotide sequences. Modulators of leupaxin activity may be therapeutically useful in treatment of a wide range of diseases and physiological conditions in which leupaxin biological activity is known to be involved.

### DETAILED DESCRIPTION OF THE INVENTION

The present invention is illustrated by the following examples. Example 1 describes isolation of a cDNA encoding leupaxin, and the encoded leupaxin polypeptide encoded is characterized in Example 2. Example 3 describes Northern analysis of leupaxin expression. Example 4 characterizes subcellular localization of leupaxin in different cell types. Example 5 demonstrates that leupaxin is a tyrosine kinase substrate. Example 6 relates to identification of a putative binding partner of leupaxin using immunoprecipitation. Example 7 relates to leupaxin participation in chemotaxis. Example 8 describes isolation of a mouse genomic clone encoding a human leupaxin species homolog. Example 9 demonstrates generation of monoclonal antibodies immunospecific for human leupaxin. Example 10 describes expression of human leupaxin. Example 11 demonstrates phosphorylation of leupaxin following integrin-mediated adhesion of cells transfected with human leupaxin cDNA. Example 12 characterizes leupaxin interaction with Pyk2. Example 13 relates to changes in cellular morphology and adhesive properties following overexpression of leupaxin. Example 14 relates to isolation of a human genomic clone and chromosomal localization. Example 15 describes expression of human leupaxin in bone.

### EXAMPLE 1

### Isolation of Leupaxin cDNA

In order to identify novel proteins expressed by macrophages, a random sequencing screen of a human macrophage cDNA library was carried out. Initially, a unique 1.2 kb clone cDNA was identified encoding an incomplete coding region homologous to the 3′ region of paxillin. The sequence of the 5′ terminus for the 1.2 kb clone is set out in SEQ ID NO: 3, and the sequence for the 3′ terminus is set out in SEQ ID NO: 8. In an attempt to identify the full length clone, the 1.2 kb cDNA was used as a probe to screen a spleen cDNA library as follows.

The 1.2 kb insert was labeled with ³²P using a Random Primed Labeling Kit (Boehringer Mannheim) and used as a hybridization probe to screen oligo(dT)-primed double-stranded cDNA prepared from poly(A)⁺ RNA isolated from normal human spleen. The library was constructed by adding BstX1 linkers to isolated cDNA and cloning the resulting cDNA into the vector pcDNA1/Amp (InVitrogen). Labeled probe was added to colony replicas prepared by standard techniques in hybridization buffer (5×SSC, 5×Denhardt's, 1% SDS and 45% formamide) and hybridization was carried out overnight at 42° C. The final wash in buffer containing 0.5×SSC and 0.1% SDS was carried out at 50° C.

Two 1.9 kb cDNAs were isolated, sequenced, and found to be identical over both complete coding regions. Each clone contained an open reading frame encoding 385 amino acid residues and a 5′ translational start codon in the context of a consensus KOZAK sequence. In a BLASTP search of the National Center for Biotechnology Information (NCBI) database, the deduced amino acid sequence for the clone was found to be most homologous to paxillin. Because the protein sequence appeared to be related to paxillin and expressed preferentially in leukocytes (discussed below), it was designated leupaxin. The polynucleotide and amino acid sequences of leupaxin are set out in SEQ ID NOs: 1 and 2, respectively.

### EXAMPLE 2

### Characterization of Leupaxin

The overall amino acid sequence identity between leupaxin and paxillin was determined to be 37%, however, the carboxy terminal regions of the proteins, leupaxin residues 151-385, showed 70% identity and 80% similarity. The conserved region common to leupaxin and paxillin was found to contain four LIM domains; homology between the four domains ranged from 67% to 76% identity. Leupaxin LIM domains contain two zinc finger motifs with a consensus sequence. As discussed supra, LIM domains have been implicated in protein binding and/or localization and in paxillin, LIM3 has been shown to mediate localization to focal adhesions and LIM2 appears to cooperate in this localization [Brown, et al., J. Cell Biol. 135:1109-1123 (1996)] though the focal adhesion ligand for paxillin LIM3 has not yet been identified. In view of the sequence similarity between leupaxin and paxillin, leupaxin LIM domains may also function in localization to focal contacts.

The amino-terminal region of leupaxin is shorter than the corresponding region in paxillin and exhibits low sequence homology except for three short regions of approximately thirteen amino acids. Residues 1-15, 85-102 and 127-149 of leupaxin as defined in SEQ ID NO: 2 share 53%, 56% and 63% identity with the corresponding regions in paxillin. When conservative substitutions are taken into consideration, similarity between the sequences is 90%, 72% and 75%. These regions in paxillin are designated LD sequences because each contains the characteristic leucine and aspartate dipeptide pair near the amino terminus [Brown, et al., J. Cell Biol. 135:1109-1123 (1996)]. The leupaxin LD sequences align with regions in paxillin designated LD 1, LD3 and LD4. The same three leupaxin LD regions can also be aligned with the LD regions identified in Hic-5, which like paxillin, has four identifiable LD domains. Leupaxin also includes a potential fourth LD motif at residues 39-51 that contains three invariable residues found in the LD2 domains of both paxillin and Hic-5. This additional potential LD domain is more closely related to the Hic-5 sequence, showing little homology to paxillin LD2. In the absence of a paxillin LD2 motif, leupaxin binding to either FAK or vinculin would therefore be predicted to differ from that of paxillin. Consistent with this prediction, preliminary results have failed to identify FAK in leupaxin immunoprecipitates from lymphoid cells. It is likely that leupaxin may interact with one or more other cytoplasmic proteins, possibly including other paxillin ligands such as src [Glenney, et al., J. Cell Biol. 108:2401-2408 (1989)], Csk [Sabe, et al., Proc. Natl. Acad. Sci. USA. 91:3984-3988 (1994)], Lyn [Minoguchi, et al., Mol. Immunol. 31:519-529 (1994)], crk [Birge, et al., Mol. Cell. Biol. 13:4648-4656 (1993)] and/or talin, [Turner, et al., J. Cell. Biol. 111:1059-1068 (1990)].

In view of the known interactions between paxillin and other cytoplasmic proteins through LIM and LD domain sequences, and the similar sequences found in leupaxin, it is likely that leupaxin may also serve an adapter function and localize cytoplasmic molecules to specific subcellular locations.

### EXAMPLE 3

### Northern Analysis of Leupaxin Expression

In order to determine the range of cell types and tissues that express leupaxin, leupaxin cDNA was used to probe blots of mRNA isolated from various sources.

A probe labeled by random priming containing the entire leupaxin coding region was used to hybridize to Human Cancer Cell Line, Human Immune System Northern and Human Multiple Tissue Northern blots (Clontech). Northern blots were hybridized at 68° C. for 1 hour in ExpressHyb Solution (Clontech) and blots were washed to a final stringency of 0.1×SSC/0.1% SDS at 50° C.

Consistent with the size of the leupaxin cDNA, the probe hybridized to a 2.4 kb mRNA present in lymphoid tissues including spleen, lymph node, thymus, and appendix. Markedly less leupaxin mRNA was detected in lung, bone marrow, fetal liver, and pancreas, and virtually none detected in heart, brain, placenta, adult liver, skeletal muscle, and kidney. Leupaxin mRNA was also detected in peripheral blood lymphocytes and the hematopoietic cell lines HL60, Molt4, and Raji cells, and to a lesser extent in K562 cells. Leupaxin mRNA in four different epithelial cell lines was detected at levels similar to that observed in K562 cells.

Leupaxin mRNA levels therefore appeared to be markedly higher in lymphoid tissues and certain hematopoietic cell lines relative to non-hematopoietic cell types. In addition, bone marrow cells appeared to contain low levels of leupaxin mRNA relative to lymphocytes and lymphoid tissues suggesting that leupaxin expression in hematopoietic cells may increase with differentiation.

### EXAMPLE 4

### Expression and Subcellular Localization of Leupaxin

In order to further characterize leupaxin, and more particularly to determine subcellular localization of the protein, leupaxin was expressed as an enhanced green fluorescent protein (EGFP) chimeric protein in a lymphoblastoid line, JY8, and in CHO fibroblast cells.

The entire leupaxin coding region was ligated in reading frame to the 3′ terminus of the EGFP coding sequence previously isolated from the vector pEGFP-C1 (Clontech). The resulting EGFP-leupaxin chimeric DNA was inserted into vector pCEP4 (InVitrogen, Carlsbad, Calif.) and the resulting plasmid, pEGFP-PX2-CEP4, transfected by electroporation into either a JY cell line previously transfected to stably express the IL-8 receptor or CHO cells. Transfectants were selected by culturing in media containing 0.5 mg/ml of Hygromycin B. EGFP-leupaxin JY8 transfectants were placed on coverslips coated with ICAM-1-Ig and allowed to adhere for 45 minutes at 37° C. Bound cells were fixed in 3% paraformaldehyde, washed with Dulbeccos PBS (D-PBS) and then counterstained with rhodamine phalloidin (Molecular Probes, Eugene, Oreg.) for 30 minutes at room temperature. Coverslips were washed with D-PBS and mounted in N-propyl-gallate (NPG). Cells were visualized with Deltavision using a CCD camera to detect images through a Zeiss Axiovert microscope. Image blur is corrected computationally using constrained iterative deconvolution algorithms. To facilitate determination of leupaxin localization in JY8 cells, the assay was carried out with cells that were adherent to, and spread on, an ICAM-1 coated substrate. ICAM-1 is a ligand for the only β₂ integrin, α_{L}β₂, expressed in JY8 cells.

In JY8 cells, leupaxin was found to be diffusely distributed in the f-actin rich cortical cytoskeleton that stains with rhodamine-phalloidin and in a region adjacent to the f-actin rich cortical cytoplasm. Leupaxin was also detected in proximal regions of filipodia-like projections, but was excluded, or at least detected at a much lower level, in the distal tips of the projections. In general, GFP-leupaxin was diffusely distributed throughout the nucleus and cytoplasm. In addition, it was unclear if leupaxin was present in adherent cell focal adhesions. Because it has previously been suggested that focal adhesions in motile cells, such as leukocytes, are relatively diffuse, much smaller, greater in number, and more uniformly distributed than in adherent cells, association of leupaxin with focal adhesions cannot be ruled out on the basis of heterologous expression alone.

In a second experiment, using a non-hematopoietic cell type, DG44 CHO cells (ATCC) were transfected with pEGFP-PX2-CEP4 by electroporation and stable expressing cells were selected in hygromycin B (700 μg/ml). Coverslips were coated overnight with 0.1 mg/ml human fibronectin (Sigma) at 4° C. Cells were trypsinized and plated in serum-free medium on coated coverslips. Sixteen hours later, cells were fixed in 3.7% paraformaldehyde in D-PBS for 8 minutes, rinsed in PBS, and permeabilized for 2 minutes in CSK buffer (100 mM NaCl, 300 mM sucrose, 3 mM MgCl₂, 0.5% Triton X-100, 10 mM Pipes pH 6.8). The coverslips were then incubated at room temperature with the anti-phosphotyrosine antibody py20 at 10 μg/ml (Transduction Labs) for 45 minutes, rinsed in PBS and incubated with lissamine-conjugated F(ab′)₂ goat-anti-mouse antibody at 10 μg/ml (Jackson Labs) for 45 minutes, rinsed in PBS, and visualized by confocal microscopy using a BioRad confocal microscope station.

In CHO cells, EGFP-leupaxin localized to distinct foci, which, like focal adhesions, are enriched with vinculin and proteins phosphorylated on tyrosine residues.

Overall, these results indicate that, in non-hematopoietic CHO fibroblasts, the leupaxin-GFP fusion protein localized to focal contacts, while in the lymphoid cells, protein localization was relatively diffuse. These observations are consistent with previous reports suggesting relatively diffuse adhesive sites in lymphoid cells and discrete focal contacts in non-hematopoietic cells [Kolega, et al., J. Cell Sci. 54:23-34 (1982)].

While it is unclear which sequences in leupaxin participate in localization, one LIM domain in leupaxin is approximately 70% identical to paxillin LIM3 at the amino acid level. Because LIM3 appears to be essential for paxillin localization to focal contacts [Brown, et al., J. Cell Biol. 135:1109-1123 (1996)], it is possible that leupaxin localization is mediated through the corresponding homologous sequence and possibly through interaction with the same, or a similar, ligand that mediates localization of paxillin. To date, no paxillin ligand that localizes the protein to focal contacts has been identified, but candidate ligands could include proteins having LIM-interacting motifs, proteins with other LIM domains, or integral membrane proteins with tyrosine-containing tight turn motifs [Wu and Gill, J. Biol. Chem. 269:25085-25090 (1994)].

### EXAMPLE 5

### Detection of Tyrosine Phosphorylated Leupaxin

In order to determine if leupaxin is a tyrosine kinase substrate, the EGFP-leupaxin chimera was expressed in JY8 cells, immunoprecipitated from cell lysate using anti-EGFP polyclonal antisera, and tested for reactivity with a phosphotyrosine specific monoclonal antibody. The rationale for this examination was based on the knowledge that (i) tyrosine phosphorylated proteins such as paxillin concentrate in focal adhesions and are likely to mediate signaling following integrin engagement, (ii) leupaxin contains at least ten potential tyrosine phosphorylation sites, and (iii) leupaxin was shown above to localize with tyrosine phosphorylated proteins in focal adhesions in nonlymphoid CHO cells.

EGFP-leupaxin protein was immunoprecipitated from JY8 transfectants (expressed as described in Example 4) using EGFP polyclonal antisera (Clontech). Approximately 40×10⁶ cells were lysed in 1.5 ml 1% CHAPS lysis buffer containing 0.01 mg/ml each soybean trypsin inhibitor (SBTI), aprotinin, and leupeptin, 1 mM 4-(2-aminoethyl) benzenesulfonyl fluoride (AEBSF), and 2 mM Na₃VO₄. Immunoprecipitated EGFP and EGFP-leupaxin were separated on a 12% Tris-glycine Novex gel and transferred to a PVDF membrane by standard techniques. Tyrosine phosphorylation was determined by Western blotting with the anti-phosphotyrosine monoclonal antibody RC20H (Transduction Labs). Phenylphosphate (0.5 mM was preincubated with RC20H for 40 minutes at 4° C. in inhibition studies. As a negative control, EGFP was analyzed in parallel.

The EGFP-leupaxin fusion migrated as a 78 kD protein whereas EGFP migrated at approximately 33 kD. The difference in observed molecular weight (45 kD) was consistent with the predicted size of leupaxin. The EGFP-leupaxin band bound the anti-phosphotyrosine antibody and the binding was inhibited with phenylphosphate, thereby confirming specificity of the binding. No antibody binding was detected with EGFP alone, suggesting that leupaxin is a tyrosine kinase substrate.

The results suggest that leupaxin function may be regulated by a tyrosine kinase activity which is consistent with the previous demonstration that paxillin is phosphorylated on tyrosine and that phosphorylation can be induced with cell adhesion or engagement of integrins with monoclonal antibodies [Turner and Miller, J. Cell Sci. 107:1583-1591 (1994); Burridge, et al., J. Cell Biol. 119:893-903 (1992); and Fuortes, et al., J. Cell Biol. 127:1477-1483 (1994)], cell transformation [Glenney and Zokas, J. Cell Biol. 108:2401-2408 (1989)], and in response to mitogens and signaling through G-protein coupled seven transmembrane receptors [Fuortes, et al., J. Cell Biol. 127:1477-1483 (1994); Zachary, et al., J. Biol. Chem. 268:22060-22065 (1993)]. Thus tyrosine phosphorylation of paxillin occurs in the induction and process of cell adhesion, motility, and growth.

### EXAMPLE 6

### Co-immunoprecipitation of PYK2 and Leupaxin

In order to assess the possible association of leupaxin with PYK2, proteins in cell lysate from JY8 cells transformed as described above were immunoprecipitated using anti-PYK2 polyclonal antisera.

Briefly, JY8 GFP or GFP-leupaxin transfectants (20×10⁶ cells) were lysed in 1 ml 1% CHAPS lysis buffer containing protease inhibitors (complete-EDTA inhibitor tablet, Boehringer Mannheim) and 1 ml Na₃VO₄. Approximately 5 μg anti-PYK2 polyclonal antibody was added and bound proteins immunoprecipitated according to standard techniques. Proteins were also immunoprecipitated using anti-GFP polyclonal antisera (Clontech). Immunoprecipitated proteins were separated by electrophoresis using a 12% Tris-glycine (Novex) gel and following resolution, proteins were transferred to a PVDF membrane. Western blotting was carried out by standard techniques using an anti-GFP monoclonal antibody (Clontech) or an anti-PYK2 monoclonal antibody, P47120 (Transduction Labs) to detect precipitated proteins.

Immunoblots demonstrated that EGFP-leupaxin co-immunoprecipitated with PYK2 from the JY8 lysate using anti-PYK2 antibody. The association appeared to be specific for leupaxin as GFP did not co-immunoprecipitate with PYK2 although similar amounts of PYK2 were immunoprecipitated from both lysates. In the reciprocal experiment, PYK2 was found to co-immunoprecipitate with GFP-leupaxin using the anti-GFP monoclonal antibody. The association was specific for leupaxin as no PYK2 was precipitated with GFP although equal amounts of GFP and GFP-leupaxin were immunoprecipitated from the two lysates. Control antibodies did not permit precipitation of either PYK2 or leupaxin.

The results suggest that leupaxin/PYK2 association may occur in leukocytes. PYK2 is in the FAK family of proteins and is expressed preferentially in leukocytes. Activity of PYK2 can be postulated to overlap those activities known for FAK, in particular, activities such as cell motility, spreading, and apoptosis. Thus, leupaxin may serve to localize PYK2 to various subcellular sites to support and modulate these biological activities. Leupaxin may therefore modulate PYK2 activity and serve a regulatory function in integrin-mediated or G protein coupled receptor transmembrane signaling.

### EXAMPLE 7

### Leupaxin Participation in Chemotaxis

In order to identify a role for leupaxin in chemotaxis, a leupaxin amino terminal fragment including LD motifs and a carboxy terminal fragment containing LIM domains were separately expressed in JY8 as GFP fusion proteins.

Briefly, leupaxin fragments were expressed in the pEGFP-CEP4 expression vector. DNA encoding the leupaxin domain from amino acid residue 2 to 150 (as set out in SEQ ID NO: 2) was amplified using PCR using the full length leupaxin clone as template DNA in a reaction with primers as set out in SEQ ID NOs: 4 and 5.

| | |
|---|---|
| ATATCTCGAGAAGAGTTAGATGCCTTATTGG | SEQ ID NO:4 |
| ATATAAGCTTTCAGCCCTTGGGCACTGTGG | SEQ ID NO:5 |

PCR conditions included 30 cycles of denaturation at 92° C. for 0.5 minutes, annealing at 42° C. for 0.5 minutes, and extension at 72° C. for 0.5 minutes. The resulting amplification product was digested with XhoI and HindIII and inserted into the expression vector previously digested with the same enzymes to give plasmid pEGFP-LD-CEP4.

DNA encoding the leupaxin LIM domain fragment from amino acid residue 145 to 386 was also amplified using PCR with the full length leupaxin cDNA as template and primer as set out in SEQ ID NOs: 6 and 7.

| | |
|---|---|
| ATATCTCGAGCCACAGTGCCCAAGGGCC | SEQ ID NO:6 |
| ATATAAGCTTTTACAGTGGGAAGAGCTT | SEQ ID NO:7 |

Reactions conditions included 30 cycles of denaturation for 30 seconds at 92° C., annealing for 30 seconds at 40° C., and extension for 30 seconds at 72° C. The amplification product was digested with XhoI and HindIII and the resulting DNA inserted in the expression vector previously digested with the same enzymes. The plasmids, pEGFP-CEP4, pEGFP-PX2-CEP4, pEGFP-LD-CEP4 and pEGFP-LIM-CEP4 were separately transfected into JY8 cells and transformants selected using media containing 0.5 mg/ml hygromycin. Expression of the individual fusion proteins was confirmed using FACS analysis and Western blotting using an anti-GFP monoclonal antibody (Boehringer Mannheim). JY8 cells found to overexpress leupaxin or a leupaxin fragment were assayed for chemotactic movement toward IL-8 on surfaces coated with ICAM-1, VCAM-1 or Vitronectin.

In the results from three assays, JY8 transfectants expressing the amino terminal leupaxin fragment demonstrated approximately 50-300% greater migration on surfaces with immobilzed VCAM-1 compared to cells expressing GFP alone suggesting a role for leupaxin in chemotaxis.

### EXAMPLE 8

### Isolation of a Mouse Genomic Leupaxin Clone

In an attempt to determine if a mouse species homolog to human leupaxin exists, a BLASTn search of the GenBank EST database was carried out using the full length human leupaxin cDNA as the query sequence. One EST, AA172749 (SEQ ID NO: 9), was identified with 89% homology to human leupaxin over 302 nucleotides, suggesting the existence of a mouse homolog. A second BLASTn search was carried out using AA172749 as the query sequence and two additional mouse clones, AA250453 (SEQ ID NO: 10) and AA177619 (SEQ ID NO: 11) were identified. A consensus sequence was determined from alignment of the AA172749, AA250453, and AA177619 sequences and the consensus sequence was used to design oligonucleotides for PCR to amplify a portion of the putative leupaxin gene from a mouse genomic DNA library. The resulting primers, PX2.M5A and PX2.M3A, are set out in SEQ ID NOs: 12 and 13, respectively.

| | | |
|---|---|---|
| PX2.M5A | ATATAAGCTTGCCGAAACGATCTTCAAGG | SEQ ID NO:12 |
| PX2.M3A | ATATGTCGACCCTGACAGCAAAGAGAGAG | SEQ ID NO:13 |

PCR was carried out using mouse genomic DNA (prepared using standard procedures) as template DNA. Samples were initially incubated at 94° C. for five minutes followed by 30 cycles of: 94° C. for 30 seconds, 55° C. for 30 seconds, and 72° C. for one minute. A final extension step was carried out at 72° C. for seven minutes. The resulting PCR product was digested with HindIII and SalI and ligated into Bluescript® vector previously digested with the same two enzymes. The resulting clone, designated mouse leupaxin/BS/2, was verified by sequencing.

The plasmid insert was amplified a second time by PCR using the primers PX2.M5A (SEQ ID NO: 12) and PX2.M3A (SEQ ID NO: 13) and the mouse leupaxin/BS/2 clone as the template in a reaction including an initial incubation at 94° C. for five minutes followed by 30 cycles of 94° C. for 30 seconds, 55° C. for 30 seconds, and 72° C. for 30 seconds. A final extension step at 72° C. for seven minutes was also carried out. The resulting PCR product was gel purified and forwarded to Genome Systems (St. Louis, Mo.) for use as a hybridization probe to screen a mouse ES-129/SvJ 1 genomic DNA BAC library (catalog #BAC-4921) in an attempt to identify a genomic fragment to be used for generating a leupaxin knockout mouse.

The BAC library screening resulted in recovery of three clones designated 19119 (clone address 304h13), 19120 (address 341d10) and 19121 (address 360h20). The clone address is a designation given by Genome Systems in order to identify the various clones. The three individual clones were digested with EcoRI, BamHI, and MspI and the fragments were separately subcloned into Bluescript®. Sequencing of the subclones allows determination of intron/exon boundaries, as well as potential alternative splice sites. Structural information relating to the genomic DNA is used for designing vectors to produce leupaxin knockout mice.

### EXAMPLE 9

### Generation of Leupaxin Monoclonal Antibodies

Leupaxin was cloned into a bacterial expression vector to produce a leupaxin polypeptide tagged with histidine repeats at each end. A sequence encoding leupaxin was amplified initially by PCR using primers that introduced restriction sites at each terminus to facilitate in frame cloning into the pBAR8 vector. The PCR reaction was carried out using full-length leupaxin cDNA as template and primers as set out in SEQ ID NOs: 14 and 15.

| | |
|---|---|
| PX2.3SAL2 | |
| ATATGTCGACCAGTGGGAAGAGCTTATTG | SEQ ID NO:14 |
| PX2.5NOT | |
| ATATGCGGCCGCGATGGAAGAGTTAGATGCC | SEQ ID NO:15 |

PCR included an initial incubation at 94° C. for six minutes followed by 30 cycles of: 94° C. for 30 seconds, 55° C. for 30 seconds and 72° C. for 30 seconds. The resulting amplification product was digested with SalI and NotI and ligated into pBAR8 previously digested with NotI and XhoI. The resulting clone was verified by sequencing and transformed into a BL21 pLysS E. coli strain (Novagen) for expression. Histidine-tagged leupaxin was isolated under denaturing conditions (in the presence of 6 M guanidine-HCI) using a nickel-charged Pharmacia HiTrap column and elution with an imidazole gradient. Maximum elution of leupaxin occurred at 150 mM and to a lesser extent at 90 mM and 250 mM.

Approximately 14 μg of the histidine-tagged leupaxin was used to immunize two mice by intrasplenic injection [Spitz, Methods in Enzymology, 121:33-41(1986)]. Ten days after the initial immunization, test bleeds from both animals showed reactivity with leupaxin in an ELISA assay carried out as follows.

An Immulon 4 plate was initially coated overnight with 3 μg/ml of histidine-tagged leupaxin in carbonate/bicarbonate buffer, pH 9.6. The plate was washed with D-PBS and blocked for 30 minutes at 37° C. with fish skin gelatin. The plate was washed again in D-PBS and a horseradish peroxidase-(HRP) conjugated goat anti-mouse secondary antibody was added at a 1:2000 dilution. Incubation was continued for 30 minutes at 37° C. The plate was washed again in D-PBS and developed with O-phenylenediamine dihydrochloride (OPD). The mouse with sera showing stronger reactivity was boosted with another 14 μg of histidine-tagged leupaxin, again by intrasplenic injection. On day 25, the spleen was removed and fusion was carried out as follows.

Briefly, a single-cell suspension was formed by grinding the spleen between the frosted ends of two glass microscope slides submerged in serum-free RPMI 1640 supplemented with 2 mM L-glutamine, 1 mM sodium pyruvate, 100 units/ml penicillin, and 100 g/ml streptomycin (RPMI) (Gibco, Canada). The cell suspension was filtered through a sterile 70-mesh Nitex cell strainer (Becton Dickinson, Parsippany, N.J.). Cells were washed twice by centrifuging at 200×g for five minutes and, after the last wash, the cell pellet was resuspended in 20 ml serum-free RPMI. Thymocytes from three naive Balb/c mice were prepared in a similar manner.

NS-1 myeloma cells, kept in log phase in RPMI with 10% fetal calf serum (Hyclone Laboratories, Inc., Logan, Utah) for three days prior to fusion, were harvested by centrifugation at 200×g for five minutes, and the cell pellet was washed twice as described above. After washing, a 10 ml suspension of the cells was prepared in serum-free RPMI and the viable cells were counted.

Spleen cells were combined with NS-1 cells in a ratio of 5:1, the mixture was centrifuged, and the supernatant was removed by aspiration. The cell pellet was dislodged by tapping the tube and two ml of 37° C. PEG 1500 (50% in 75 mM Hepes, pH 8.0) (Boehringer Mannheim) was added to the cells with stirring over the course of one minute. Fourteen ml of serum-free RPM I was then added over a seven minute time course. An additional 16 ml RPMI was added and the cells were collected by centrifugation at 200×g for ten minutes. The supernatant was removed and discarded, and the cell pellet was resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim), and 1.5×10⁶ thymocytes/ml. The suspension was dispensed into ten 96-well flat bottom tissue culture plates (Corning, United Kingdom) at 200 μl/well. Cells were fed three to five times by aspirating approximately 100 μfrom each well with a 20 gauge needle (Becton Dickinson) and adding 100 μl/well plating medium described above except containing 10 units/m IL-6 and lacking thymocytes. Supernatants from the fusions were screened initially by ELISA and Western blot, both techniques carries out using standard methods routinely practiced in the art. Clones in positive wells were subcloned three times successively to ensure a clonal population using RPMI, FBS, 100 μM sodium hypoxanthine, 16 μM thymidine, and 10 units/ml IL-6. Subcloning was performed by doubling dilution.

Supernatants from 42 wells that were positive in the primary ELISA screen were then tested by Western blotting for recognition of leupaxin and GFP-tagged leupaxin in lysate from JY8 transfectants. Lysates were obtained from JY8 cells previously transfected with the pEGFP-PX2-CEP4 expression plasmid described in Examples 4 and 5.

Supernatants from thirty of the positive wells were tested further by antibody staining of CHO cells transfected with the GFP-leupaxin expression plasmid. Briefly, CHO cells transfected with GFP-leupaxin were grown overnight in 8-chamber slides coated with fibronectin at 0.1 mg/ml. Media was washed from the wells and 100 μl of culture supernatant from each of the thirty positive wells was added for 45 minutes at room temperature. The primary antibody was removed by washing and a goat anti-mouse lissamine-conjugated secondary antibody was added at a dilution of 1:100 for 45 minutes at room temperature. The cells were washed and viewed using an inverted scope to detect co-localization of the green GFP-leupaxin signal and the red lissamine signal associated with the detection of leupaxin by the primary monoclonal antibody.

Nine hybridomas identified by Western blotting and immunohistochemistry that secreted monoclonal antibodies recognizing leupaxin were designated 283A, 283B, 283C, 283D, 283G, 283H 283J, 283N, and 283P. Antibodies in supernatant from all nine hybridomas immunoprecipitated leupaxin, with antibodies from hybridomas 283B, 283C and 283G being most efficient.

### EXAMPLE 10

### Leupaxin Expression

Expression in Cell Lines

Antibodies described above were used to examine human leupaxin expression in various cell lines by Western blotting using whole cell lysates. Lysates were prepared from approximately 2×10⁷ cells per cell line in one ml of 1% Ipegal lysis buffer (50 mM Tris, pH 7.5, 150 mM NaCl, 1 mM Na₃VO₄) containing protease inhibitors introduced in the form of one Complete minus EDTA protease inhibitor tablet (Boehringer Mannheim) per 10 ml of lysis buffer. Cells were lysed on ice for 30 minutes and the soluble fraction was recovered after centrifugation at 12,500 rpm for 10 minutes in a tabletop centrifuge. Soluble proteins were separated using gel electrophoresis and transferred to membranes according to standard procedures. Leupaxin expression was detected using standard Western blotting with anti-leupaxin monoclonal antibodies 283C and 283G.

Leupaxin expression was detected in several hematopoietic cell lines including JY8, Raji, Jurkat77, Hut78, U937, and HL60, but expression was not detected in the monotypic THP 1 cell line. Leupaxin expression was not observed in the non-lymphoid HEL, 293, or A549 cell lines. Leupaxin expression was also detected in two primary endothelial cultures, HUVEC and HUAEC, at levels somewhat lower than that observed the in lymphoid cell lines.

Expression on Peripheral Blood Leukocytes

In order to assess leupaxin expression in peripheral blood leukocytes (PBL), the following analysis was carried out using PBLs isolated from freshly drawn blood. Briefly, blood was collected in sodium citrate (3.8% w/v in dH₂O), pH 7.2, at a ratio of 10 ml of blood to 1 ml of sodium citrate solution. The blood mixture was diluted 2:3 in Dulbecco's PBS (D-PBS) solution and layered over a 1Histopaque gradient at a ratio of two parts blood to one part Histopaque. The gradient was centrifuged at room temperature at a speed of 1450 rpm (480×g) for 30 minutes and centrifugation was stopped without a brake. Plasma was removed from the top of the gradient and the peripheral blood mononuclear cells (PBMC) were collected and washed with D-PBS containing 1 mg/ml of bovine serum albumin (BSA).

For PBL isolation, isolated PBMCs were plated in a T25 flask and incubated for two hours in RPMI media containing 10% FBS at 37° C. and 5% CO₂. Non-adherent PBLs were collected, washed with D-PBS, and lysed in 1% Ipegal lysis buffer as described above.

For purification of monocytes, isolated PBMCs were resuspended in D-PBS containing 1 mg/ml of BSA and mixed in a 2:1 ratio with a previously prepared Percoll solution (10 ml of Percoll mixed with 1.65 ml of Hank's Buffered Salt Solution and the pH adjusted to 7.0 using approximately 100 μl of 1 N HCl). The Percoll gradient was centrifuged at a speed of 1690 rpm for 25 minutes in a fixed angle rotor (345×g) and centrifugation was stopped without a brake. Monocytes were collected from the top of the gradient and washed in D-PBS containing 1 mg/ml BSA. Isolated monocytes were cultured for lengths of time varying from two hours to 11 days in RPMI containing 10% serum at 37° C. and 5% CO₂. At each time point, lysates were prepared from the cells as described above, equal amounts of protein were separated using gel electrophoresis, the separated proteins were transferred to a membrane, and Western blotting was carried out using a standard protocol with a leupaxin monoclonal antibody as described above.

Leupaxin expression increased significantly in monocytes that were cultured for several days in comparison to freshly isolated monocytes or monocytes cultured for only a few hours. This result may be explained by the fact that when monocytes are maintained in culture in RPMI media with 10% serum, they differentiate into macrophage-like cells, adopting morphology similar to macrophages and displaying some of the same surface markers as found in more mature macrophages.

HL60 Cell Line Expression

HL60 is a promyelocytic leukemia cell line which can be induced to differentiate into a macrophage-like cell type by adding 16 nM PMA to the normal culture media (RPMI with 10% FBS). In order to examine possible changes in leupaxin expression during the differentiation process, HL60 cells were cultured with 16 nM PMA over time ranging from 0.5 hours to three days. After 24 hours of PMA incubation, the majority of the HL60 cells were adherent to the tissue culture plastic. At each time point, cells were collected and lysed in 1% Ipegal lysis buffer as described above. Lysate protein was separated by gel electrophoresis and transferred to a PVDF membrane using standard procedures. Western blotting was carried out by standard procedures using the anti-leupaxin monoclonal antibody 283G.

No appreciable change in leupaxin expression was detected in early time points (0.5, 1 or 3 hour incubations), but expression increased significantly after 24 hours of PMA treatment and remained at relatively constant levels for the next 48 hours. The timing of paxillin expression also appeared to be up regulated in these cells, but the up-regulation lagged that of leupaxin. Little or no expression paxillin expression was detected until 24 hours of PMA treatment and expression increased after one and two days of additional incubation.

Leupaxin Expression in Osteoclasts

Osteoclasts are derived from the same progenitor cells as macrophages. In order to assess expression of leupaxin on this cell type, monoclonal antibody 283G was used in an immunohistochemical analysis to stain frozen sections of normal and arthritic rat joints.

A normal rat joint and a rheumatoid arthritis rat joint were sectioned at 6 μm thickness and air dried on Superfrost Plus (VWR Scientific) slides for five minutes at room temperature. Slides were stored at −20° C. until the assay was performed. Prior to use, slides were incubated at 50° C. for approximately five minutes. Sections were fixed in 4° C. acetone (EM Science) for two minutes and each section was blocked for one hour at room temperature with 100 μl of a solution containing 30% normal rat serum (Harlan), and 1% BSA (Sigma) in 1×TBS. The blocking solution was removed by blotting and the sections were placed in a solution of 100 ml of 1×TBS containing 1.1 ml 30% H₂O₂ (Sigma) and 1.0 ml 10% NaN₃ (Sigma), for 15 minutes at room temperature to remove endogenous peroxidase activity. Sections were washed in 1×TBS for approximately one minute and endogenous biotin was blocked by incubating the sections for 15 minutes in 1×TBS containing one drop of avidin from an avidin/biotin blocking kit (Vector). Sections were then washed one minute in 1×TBS and 75 μl of each of 283G and 283C antibody was added separately as hybridoma supernatant. Incubation was continued for one hour at room temperature and sections were washed three times in 1×TBS for five minutes in each wash to remove unbound antibody. Excess TBS was removed by aspirating around the tissue following the final wash. Biotinylated rat anti-mouse antibody (Jackson Laboratories) was diluted 1:400 in a solution containing 3% normal human serum (BBI), 5% normal rat serum (Harlan), and 1% BSA (Sigma) in 1×TBS and 75 μl of the solution was applied to each section for 30 minutes at room temperature. Slides were washed two times in 1×TBS for five minutes each wash, after which 3,3′-diaminobenzidine (DAB) substrate (Vector Laboratories) was applied. Color development was stopped by immersion in water. Sections were counterstained in Gill's hematoxylin #2 (Sigma) and rinsed in water before dehydrating and mounting with Cytoseal (VWR).

Leupaxin expression was detected in vessels, osteoblasts, and osteoclasts in the arthritic joint, while little labeling was observed in the normal joint. The increased leupaxin expression detected may be directly attributed to the arthritic condition or indirectly attributed to the condition in view of an increased number of osteoblasts and osteoclasts present as a result of the changes in bone structure in an arthritic joint. Leupaxin expression localized to the osteoclast podosome structures, subcellular structures at which the osteoclast seals itself to the bone mineral matrix and a site to which Pyk2 localization has previously been demonstrated.

### EXAMPLE 11

### Phosphorylation of Leupaxin Following Integrin-mediated Adhesion

Paxillin has been shown to undergo increased tyrosine phosphorylation upon integrin-medicated adhesion. In order to determine if integrin-mediated adhesion leads to a similar increase in leupaxin phosphorylation, JY8 cells were allowed to adhere to ICAM-1, VCAM-1 or vitronectin through the integrin receptors LFA-1, α₄β₇ or αᵥβ₃ respectively and changes in leupaxin phosphorylation determined.

Briefly, 10 cm tissue culture dishes were coated at 4° C. overnight with 10 μg/ml of ICAM-1, 10 μg/ml ICAM-1, or 2 μg/ml of vitronectin in bicarbonate buffer. The plates were washed with RPMI media containing 10% FBS, after which 2×10⁶ JY8 cells in 10 ml media were added to each dish. The cells were allowed to adhere during a 30 minute incubation at 37° C., after which unbound cells were removed with washing in D-PBS. The remaining adherent cells were lysed on the plate in 0.5 ml of 1% Ipegal lysis buffer (described above). Lysates were incubated on ice for 30 minutes, centrifuged for 10 minutes at 12, 500 rpm, and precleared by incubation with 30 μl of 1:1 slurry of Protein A agarose beads at 4° C. for one hour with rotation. The beads were removed by centrifugation and either 50 μl of hybridoma 283C culture supernatant or 2 μl (approximately 2 μg total protein) of a mouse isotype matched IgGl control monoclonal antibody were added for each immunoprecipitation. After a two hour incubation at 4° C. with rotation, 30 μl of Protein A agarose conjugated with rabbit anti-mouse IgG was added and incubation continued for two hours at 4° C. with rotation. The beads were collected by centrifugation, washed four times with 500 μl of 1% Ipegal lysis buffer, and resuspended in 2×SDS sample buffer. Samples were boiled for three minutes before loading on a 8% Tris-glycine gel (Novex). The separated proteins were transferred to a PVDF membrane by semi-dry blotting and analyzed by Western blotting with the anti-phosphotyrosine antibody RC20H (Transduction Labs).

Leupaxin immunoprecipitated from JY8 cells bound to immobilized ICAM-1, VCAM-1, or vitronectin showed higher levels of tyrosine phosphorylation than leupaxin immunoprecipitated from cells maintained in suspension. This result indicated that integrin-mediated adhesion leads directly or indirectly to increased leupaxin phosphorylation.

### EXAMPLE 12

### Leupaxin Interaction with Pyk2

As described above, Pyk2 is a binding partner of leupaxin. In order to determine what domains of leupaxin participate in Pyk2 binding, various regions of the leupaxin polypeptide were isolated and immobilized on agarose beads. The leupaxin-coated beads were incubated with a lysate of cells containing Pyk2 in order to determine by Western blotting if the isolated fragments would bind Pyk2. The leupaxin fragments were prepared as follows.

A polynucleotide encoding amino acid residues 2 through 149 of the human leupaxin polypeptide, a region including all four LD motifs, was amplified by PCR and subcloned into the GST fusion vector pGEX-4T-1 (Pharmacia). PCR was carried out using 1 μl each of primers Eco4F (SEQ ID NO: 16) and Xho 447R (SEQ ID NO: 17) in a 50 μl reaction including 100 ng of full length leupaxin cDNA (described in Example 1) as template, 2.5 Units Pfu polymerase (Stratagene), dNTPs, and buffer.

| | |
|---|---|
| Primer Eco4F | |
| ATAGAATTCGAAGAGTTAGATGCCTTA | SEQ ID NO:16 |
| Xho 447R | |
| ATACTCGAGCTTGGGCACTGTGGCAAT | SEQ ID NO:17 |

Amplification was carried out in 30 cycles of 94° C. for 30 seconds, 42° C. for 30 seconds, and 72° C. for two minutes. The resulting amplification product was digested with EcoR1 and XhoI at restriction cites introduced by the PCR primer (underlined as set out above) and cloned into pGEX-4T-1 previously digested with the same enzymes. The resulting expression plasmid was designated pGST-LD. Sequencing confirmed that the isolated clone contained the human cDNA sequence encoding amino acids 2 through 149 of leupaxin in frame with the glutathione-S-transferase (GST) coding sequence of the vector.

The pGST-LD expression vector was then used as template DNA in order to introduce individual mutations in each of the LD domains of leupaxin. Mutations were generated by site-directed mutagenesis using a Quick-Change mutagenesis kit (Stratagene) according to the manufacturer's instructions. The first LD motif was mutagenized using an oligomer (SEQ ID NO: 18), and its complement, which changed wild type nucleotides to the underlined nucleotides in SEQ ID NO: 18 below. The leupaxin amino acid sequence was altered from EELDALL (SEQ ID NO: 19) to EEAAAALL (SEQ ID NO: 20)

TCCCCGGAATTCTAAGCGGCAGCTGCTTATTGGAGGAAC SEQ ID NO: 18

The second LD motif was mutagenized using another oligomer (SEQ ID NO: 21), and its complement, which changed wild type leupaxin nucleotides to the underlined nucleotides shown in SEQ ID NO: 21 below. The encoded leupaxin amino acids were changed from NLDETS (SEQ ID NO: 22) to NLAAAS (SEQ ID NO: 23).

AAGGAGACTAACCTTGCTGCGGCTTCGGAGATCCTTTCT SEQ ID NO: 21

The third LD motif was mutagenized using another oligomer (SEQ ID NO: 24), and its complement, the wild type leupaxin nucleotide being changed to the nucleotides underlined in SEQ ID NO: 24 below. The leupaxin amino acid sequence was modified from QLDELM (SEQ ID NO: 25) to QAAALM (SEQ ID NO: 26).

GTCAGCAGCTGCTCAGGCGGCTGCGCTCATGGCTCACCTG SEQ ID NO: 24

The fourth LD motif was mutagenized using still another oligomer (SEQ ID NO: 27), and its complement, which changed the wild type leupaxin coding region to include the nucleotides underlined in SEQ ID NO: 27 below. The encoded leupaxin amino acid sequence was changed from SLDSM (SEQ ID NO: 28) to SAAAS (SEQ ID NO: 29).

GATCACAAGGCCTCCGCGGCCGCAATGCTTGGGGGTCTG SEQ ID NO: 27

Each mutation was confirmed by sequencing.

A polynucleotide sequence encoding leupaxin amino acid residues 92 through 106 and containing the 3^{rd} LD domain was also constructed and subcloned between the unique EcoRI and XhoI sites in vector pGEX-4T-1. PCR was carried out in a 50 μl reaction including 1 μM each of primer R1-92-F and primer Xho106-R with 100 ng of pCNA-PX2 as template DNA, 2.5 units Pfu polymerase (Stratagene), dNTPs, and buffer.

| | |
|---|---|
| R1-92-F | |
| ATATGAATTCGCTCAGTTGGATGAGCTC | SEQ ID NO:30 |
| Xho106-R | |
| ATATCTCGAGTCAAGCATCTGCTCACTGC | SEQ ID NO:31 |

Amplification was carried out in 30 cycles of 94° C. for 30 seconds, 42° C. for 30 seconds, and 72° C. for two minutes. The resulting amplification product was digested with EcoRI and XhoI at restriction sites introduced by the PCR primers (underlined in the sequences set out above). The digestion product was cloned into plasmid pGEX-4T-1 previously digested with the same two enzymes to provide plasmid pGST-92-106. Sequencing confirmed that the isolated clone contained the human leupaxin cDNA sequence encoding amino acids 92 through 106 of leupaxin (SEQ ID NO: 32) in frame with the glutathione-S-transferase (GST) coding sequence of the vector.

AQLDELMAHLTEMQA SEQ ID NO: 32

Plasmids encoding each of the GST protein, the GST-LD fusion protein, the GST-LD fusion proteins with mutations in each LD motif, and the GST-92-106 protein were transformed into E. coli by standard methods and protein production was induced in exponentially growing cultures by addition of 0.5 mM IPTG for two hours. Cells were collected by centrifugation and lysed by sonication in D-PBS with 1 mM PMSF and 1% Triton-X-100. Clarified bacterial supernatant was incubated with glutathione agarose (Pharmacia) for 20 minutes at 4° C., after which the resin was washed in D-PBS/1% Triton-X-100. Each of the various proteins was thereby immobilized on the resin.

In preparation of cell lysate including Pyk2, JY8-8 cell lysate was prepared by disrupting 4×10⁷ exponentially growing cells in one ml of Ipegal Lysis Buffer (described above) for 10 minutes on ice. Lysate was clarified by centrifugation at 10,000 rpm in a microfuge at 4° C. JY8-8 lysate (corresponding to approximately 1×10⁷ cells) was incubated for 90 minutes with 30 μl glutathione agarose on which approximately one μg of GST or GST-containing fusion protein was immobilized. The resin was washed three times with one ml Ipegal lysis buffer. Bound proteins were immunoblotted with antibody specific for Pyk2 (Transduction Labs P47120) according to the supplier's suggested protocol.

Pyk2 present in lysates of JY8-8 cells bound specifically to GST-LD protein but not to GST alone. Pyk2 binding was detected with wild-type GST-LD, and GST-LD proteins with mutations in the second or fourth LD motifs. Pyk2 showed reduced binding to GST-LD with a mutation in the first LD motif and no binding to GST-LD with a mutation in the third LD motif The results indicated that the third LD motif was required for interaction with Pyk2. Consistent with this observation, Pyk2 binding was also detected with the fusion protein encoding leupaxin amino acid residues 92 to 106, encompassing the third LD motif, but not with GST alone.

### EXAMPLE 13

### Overexpression of Leupaxin in 293T Cells

As discussed, leupaxin is related to paxillin, a protein found in focal adhesion complexes that coordinate cell adhesion and migration. In view of the leupaxin/paxillin relationship, the morphology and adhesive properties of human 293T cells (ATCC) overexpressing leupaxin were investigated. Expression vectors encoding full length or truncated leupaxin polypeptides fused to green fluorescent protein (GFP) were constructed in the mammalian expression vector, pCEP4 (Invitrogen). Plasmids were generated which encoded fusion proteins consisting of EGFP coding sequences (derived from pEGFP-C3, Clontech) (i) fused in frame to a leupaxin sequence encoding amino acid residues 1 through 386 to generate plasmid pEGFP-PX2-CEP4, (ii) fused to a leupaxin sequence encoding amino acid residues 2 through 145 to generate plasmid pCEP4-EGFP-LD, and (iii) fused to a leupaxin sequence encoding amino acid residues 145 through 386 to generate plasmid pCEP4-EGFP-LIM. A control expression plasmid, designated pCEP4-EGFP, was constructed encoding only EGFP. Plasmids were constructed as follows.

Plasmid pCEP4-EGFP encoded the enhanced green fluorescent protein (EGFP) inserted into the mammalian expression vector pCEP (Invitrogen). The EGFP coding sequence was excised from pEGFP-C1 (Clontech) by digestion with NheI and BamHI and the purified fragment was inserted into pCEP4 previously digested with the same two enzymes.

Plasmid pEGFP-PX2-CEP4 encoded full length leupaxin in frame with the 3′ end of sequences encoding EGFP. The leupaxin coding region was amplified by PCR using primers PRP/95X (SEQ ID NO. 33) and PRP/1240H (SEQ ID NO: 34) which introduce 5′ XhoI and 3′ HindIII sites, respectively, in the amplification product.

| | |
|---|---|
| PRP/95X | |
| CCCATATCTCGAGCAATGGAAGAGTTAGATGCC | SEQ ID NO:33 |
| PRP/1240H | |
| CCCATAAAGCTTTTACAGTGGGAAGAGCTT | SEQ ID NO:34 |

PCR was carried out using 1 μg of each primer in a 50 μl reaction including 100 ng template DNA comprising the full length leupaxin clone PX2/pCDNA1/2 described in Example 1, 2.5 units Pfu polymerase (Stratagene), dNTPs, and buffer. Amplification was carried out in 30 cycles of 94° C. for one minute, 55° C. for one minute, and 72° C. for one minute. The resulting amplification product was digested with XhoI and HindIII and ligated into pCEP4-EGFP previously digested with the same two enzymes.

Plasmid pCEP4-EGFP-LD encoded EGFP in frame with a human leupaxin sequence encoding amino acids 2 through 145 which includes all four LD domains. The sequence encoding the leupaxin fragment was amplified by PCR using primers that introduced an XhoI site immediately 5′ to the second amino acid codon (primer LD F, SEQ ID NO: 35, XhoI site underlined) and introduced a termination signal and a HindIII site 3′ to the codon for amino acid residue 145 (primer LD-R3, SEQ ID NO: 36, HindIII site underlined).

| | |
|---|---|
| LD F | |
| ATATCTCGAGAAGAGTTAGATGCCTTATTGG | SEQ ID NO:35 |
| LD-R3 | |
| ATATAAGCTTTCAGCCCTTGGGCACTGTG | SEQ ID NO:36 |

PCR was carried out as described above. The resulting amplification product was digested with XhoI and HindIII and ligated into pCEP4-EGFP previously digested with the same two enzymes.

Plasmid pCEP4-EGFP-LIM encoded EGFP in frame with human leupaxin cDNA encoding amino acids 145 through 386 that includes all four LIM domains. PCR was used to amplify the leupaxin region with primer LIM F (SEQ ID NO: 37), which introduces an XhoI site (underlined below), and H3 LIM R (SEQ ID NO: 38), which introduces a HindIII site (underlined below).

| | |
|---|---|
| LIM F | |
| ATATCTCGAGGCCACAGTGCCCAAG | SEQ ID NO:37 |
| H3 LIM R | |
| ATATAAGCTTTTACAGTGGGAAGAGCTT | SEQ ID NO:38 |

PCR was carried out as described above. The resulting amplification product was digested with XhoI and HindIII and ligated into pCEP4-EGFP previously digested with the same two enzymes.

Individual plasmids were transfected into 293T cells using the calcium-phosphate method, after which cells were incubated for 24 hours in 3% CO₂ at 37° C.

The pCEP4-EGFP transfectants expressed EGFP throughout the cell and exhibited a flat, adherent morphology. The pCEP4-EGFP-LD transfected cells expressed EGFP-LD fusion protein in the cytoplasm and were adherent to the plastic. The pCEP4-EGFP-LIM transfected cells expressed GST-LIM fusion protein in the cytoplasm with dense cytoplasmic collections observed in some of the cells. These cells were largely adherent to plastic. The pEGFP-PX2-CEP4 transfected cells expressed the EGFP-leupaxin chimeric protein in the cytoplasm. Cells with highest detectable expression were rounded and appeared to adhere to the substrate poorly.

Cell substratum adherence was measured using light microscopy. The transfected cells were removed from the culture dish using Versene in a five minute incubation at 37° C. The cells were passed through 0.75 μM mesh (Nitex), centrifuged the cells for five minutes, and resuspended in MEM-10PSQ at a density of 6×10⁵ cells/ml. The cells were plated on permanox slides (Nunc) previously coated with five μg/ml human fibronectin (Sigma) during a preliminary 20 minutes incubation at 37° C. Cells were fixed with 4% paraformaldehyde and examined using fluorescence microscopy. Single fluorescent cells were scored under phase contrast microscopy as either flat (phase dull) or round (phase bright).

The pCEP4-EGFP transfected cells adhered quickly, with 70% of all cells identified as being flat after 20 minutes. The 20 minute time point was selected on the basis of control experiments that were carried out to determine the time course of flattening for untransfected cells. In contrast, only 6% of the pEGFP-PX2-CEP4 transfected cells were flat after 20 minutes. The results indicated that expression of EGFP-PX2 either provoked the 293T cells to round up or inhibited cell flattening. Since fibronectin is a ligand for the integrins found on 293T cells, leupaxin may regulate integrin-dependent adhesion.

The adherence of transfected 293T cells was further examined by plating 1×10⁵ cells transfected either with pCEP4-EGFP or pEGFP-PX2-CEP4 on 96-well plates coated with fibronectin. After a 10 minute incubation at 37° C., the cells were washed gently by immersion in D-PBS at room temperature to remove non-adherent cells. The number of adherent transfected cells was measured using a micro-fluorimeter to determine fluorescence remaining in the well.

Fluorescence from the pEGFP-PX2-CEP4 transfected cells was found to be 50% of that measured for the pCEP4-EGFP transfected cells, suggesting that cells overexpressing leupaxin cells adhere poorly to the integrin ligand fibronectin.

### EXAMPLE 14

### Isolation of Human Leupaxin Genomic Clones

A clone containing the human leupaxin cDNA coding region was forwarded to Genome Systems for use as a hybridization probe to screen a human PAC genomic DNA library (catalog # PAC-6541) in an attempt to isolate a genomic sequence encoding human leupaxin. The screening resulted in recovery of two clones, designated 20316 (clone address 219(m12)) and 20317 (address 153(e13)). Sequences of the clones were verified, after which clone 20316 was returned to Genome Systems for use in fluorescent in situ hybridization (FISH) to determine the chromosomal localization of the human leupaxin gene.

Results localized leupaxin human to chromosome 11 at position 11q12, a region that has been implicated in a number of disorders. For example, in atopic hypersensitive individuals, asthma, hay fever, and eczema are common indications and these individuals are prone to produce particularly high levels of IgE in response to minute quantities of antigen. In assessing chromosomal abnormalities associated with the disorder, Jeffreys, et al., Nature 314:67-73 (1985) have reported linkage to a hypervariable minisatellite probe assigned to the chromosome 11q12-q13 region.

As another example, the bone disorder osteopetrosis is characterized by macrocephaly, progressive deafness and blindness, hepatosplenomagaly and severe anemia. Blindness is believed to arise from primary retinal atrophy. Hepatosplenomegaly is believed to result from compensatory extramedullary hematopoiesis while anemia is thought to occur as a result of bone encroachment on marrow. These last two conditions may result from defective resorption of immature bone. Mice that are homozygous for op and display osteopetrosis-like phenotype exhibit severe deficiency of osteoclasts and macrophages, however, in vitro, progenitor cells taken from osteopetrosic mice are capable of differentiating to mature macrophages. This observation suggests a deficiency in the homozygous mouse in production of macrophage growth factors or enhanced production of macrophage growth inhibitors. Results have shown that bone marrow transplant can alleviate problems associated with the disorder, presumably by providing functional cells in a localized environment conducive to differentiation and/or recruitment of osteoclasts and macrophages. Heaney, et al., [Am. J. Hum. Genet. 61 (suppl):A12 only (1997) and Hum Molec. Genet. 7:1407-1410 (1998)] have reported a novel 12-transmembrane transport protein that may be involved in preventing development of the osteopetrosic condition and linkage studies have mapped the gene locus to chromosome region 11q12-q13.

Still another bone disorder, osteoporosis-pseudoglioma syndrome (OPS), is characterized by severe osteogenesis imperfecta and blindness associated with hyperplasia of the vitreous, corneal opacity and secondary glaucoma. In addition to the visual disorders, individuals suffering from OPS tend to experience multiple bone fractures and may exhibit skeletal deformities. Linkage analysis on DNA samples from OPS individuals has led to assignment of the OPS locus to the 11q12-q13 chromosomal region [Gong et al., Am. J. Hum. Genet. 59:146-151 (1996)].

In yet another bone disorder, linkage studies in a family with very high spinal bone-mineral density indicated the presence of markers in the 11q12-q13 locus [Johnson, et al., Am. J. Hum. Genet. 60:1326-1332 (1997)]. The disorder is referred to as spinal Z(BMD). High bone mass has been shown to arise from osteosclerosis (i.e., increased density of spongy bone) and/or hyperostosis (i e., thickening of compact bone) and can occur localized in, or throughout, the skeleton.

In view of the locus identified for human leupaxin and the number of disorders associated therewith, the leupaxin gene product may be involved in any or all of the disorders having markers assigned to the 11q12 locus.

### EXAMPLE 15

### Leupaxin Expression in Human Bone

Based on the previous results observed in staining rat joint sections, experiments were designed to assess expression of the gene product in human bone.

Paraffin embedded human bone sections were obtained and paraffin was removed by subjecting the sections to serial washes including xylene (VRW), 100% ethanol (VRW), 70% ethanol and deionized water. Sections were placed in Dako Antigen Retrieval solution for twenty minutes at room temperature and endogenous peroxidase activity was removed from the sections by washing in a solution of 100 ml 1×TBS, 1.1 ml 30% H202 (Sigma), 1.0 ml NaN3 (Sigma) for 15 minutes at room temperature. Each section was blocked with 150 μl of a solution containing 30% normal human serum (Boston Biomedica) and 2% BSA (Sigma) in 1×TBS for 30 minutes at room temperature. After incubation, the solution was gently blotted from the sections. Primary monoclonal antibodies were prepared by diluting 283G and 283C supernatant at a ratio of 1:2 with blocking solution. Supernatant for hybridomas 283J and 283H was not diluted. Section were incubated with 75 μl of each antibodies preparation (diluted or undiluted) for one hour at room temperature, after which the sections were washed three times in 1×TBS for five minutes each wash to remove unbound antibody. Excess TBS was removed by aspiration. Biotinylated rat anti-mouse antibody (Jackson Laboratories) was diluted 1:400 in blocking solution and 75 μl was applied to each section for thirty minutes at room temperature. Sections were washed two times with 1×TBS for five minutes each wash. Peroxidase-conjugated avidin./biotin complex (Vector Laboratories) was prepared by adding 9 μl reagent A and 9 μl reagent B to 782 μl 1×TBS and 75 μl was applied to each section for thirty minutes at room temperature. Sections were washed two times with 1×TBS for five minutes each wash. VIP substrate (Vector Laboratories) was applied and color development was stopped by immersion of the slides in water. Sections were counterstained in methyl green (Vector Laboratories) and rinsed in water before dehydrating and mounting with Cytoseal (VWR)

Supernatant from 283J, 283G, and 283H hybridomas showed similar labeling patterns. Labeling was detected on osteoblasts, which are responsible for synthesis and secretion of the organic component in the bone extracellular matrix. Labeling was also observed on osteoclasts, which are large multi-nucleated cells involved in bone resorption. No labeling was observed with supernatant from the 283 C hybridoma or the IgG1 isotype control.

While the present invention has been described in terms of specific embodiments, it is understood that variations and modifications will occur to those skilled in the art. Accordingly, only such limitations as appear in the appended claims should be placed on the invention.

What is claimed is:

1. A purified and isolated leupaxin polypeptide.

2. The polypeptide according to claim 1 comprising the leupaxin amino acid sequence set out in SEQ ID NO: 2.

3. A polynucleotide encoding the polypeptide according to claim 1 or 2.

4. The polynucleotide according to claim 3 comprising the sequence set forth in SEQ ID NO: 1.

5. A polynucleotide encoding a human leupaxin polypeptide selected from the group consisting of:
a) the polynucleotide according to claim 2; and
b) a DNA which hybridizes under moderately stringent conditions to the complement of the polynucleotide of (a).

6. The polynucleotide of claim 5 which is a DNA molecule.

7. The DNA of claim 6 which is a cDNA molecule.

8. The DNA of claim 6 which is a genomic DNA molecule.

9. The DNA of claim 6 which is a wholly or partially chemically synthesized DNA molecule.

10. An anti-sense polynucleotide which specifically hybridizes with the complement of the polynucleotide of claim 5.

11. A expression construct comprising the polynucleotide according to claim 5.

12. A host cell transformed or transfected with the polynucleotide according to claim 11.

13. A method for producing a leupaxin polypeptide comprising the steps of:
a) growing the host cell according to claim 12 under conditions appropriate for expression of the leupaxin polypeptide and
b) isolating the leupaxin polypeptide from the host cell or the medium of its growth.

14. An antibody specifically immunoreactive with the polypeptide according to claim 1 or 2.

15. The antibody according to claim 14 which is a monoclonal antibody.

16. A hybridoma which secretes the antibody according to claim 15.

17. An anti-idiotype antibody specifically immunoreactive with the antibody according to claim 14.

18. A method to identify a specific binding partner compound of the leupaxin polypeptide according to claim 1 or 2 comprising the steps of:
a) contacting the leupaxin polypeptide with a compound under conditions which permit binding between the compound and the leupaxin polypeptide;
b) detecting binding of the compound to the leupaxin polypeptide; and
c) identifying the compound as a specific binding partner of the leupaxin polypeptide.

19. The method according to claim 18 wherein the specific binding partner modulates activity of the leupaxin polypeptide.

20. The method according to claim 19 wherein the compound inhibits activity of the leupaxin polypeptide.

21. The method according to claim 19 wherein the compound enhances activity of the leupaxin polypeptide.

22. A compound identified by the method according to claim 18.

23. A method to identify a specific binding partner compound of the leupaxin polynucleotide according to claim 5 comprising the steps of:
a) contacting the leupaxin polynucleotide with a compound under conditions which permit binding between the compound and the leupaxin polynucleotide;
b) detecting binding of the compound to the leupaxin polynucleotide; and
c) identifying the compound as a specific binding partner of the leupaxin polynucleotide.

24. The method according to claim 23 wherein the specific binding partner modulates expression of a leupaxin polypeptide encoded by the leupaxin polynucleotide.

25. The method according to claim 24 wherein the compound inhibits expression of the leupaxin polypeptide.

26. The method according to claim 24 wherein the compound enhances expression of the leupaxin polypeptide.

27. A compound identified by the method according to claim 23.

28. A composition comprising the compound according to claim 27 pharmaceutically acceptable carrier


SEQUENCE LISTING (38 sequences)

SEQ ID NO: 1 - DNA, 1875 bp, Homo sapiens
  CDS (94)..(1251)
aaagcatccagttcctttgcggtcctcttcttcagcacatgccaaagctgttcctcacggcctgtgagacaagagcatcttggatgtaggacaatggaagagttagatgccttattggaggaactggaacgctccacccttcaggacagtgatgaatattccaacccagctcctcttcccctggatcagcattccagaaaggagactaaccttgatgagacttcggagatcctttctattcaggataacacaagtcccttgccggcgcagctcgtgtatactaccaatatccaggagctcaatgtctacagtgaagcccaagagccaaaggaatcaccaccaccttctaaaacgtcagcagctgctcagttggatgagctcatggctcacctgactgagatgcaggccaaggttgcagtgagagcagatgctggcaagaagcacttaccagacaagcaggatcacaaggcctccctggactcaatgcttgggggtctggagcaggaattgcaggaccttggcattgccacagtgcccaagggccattgtgcatcctgccagaaaccgattgctgggaaggtgatccatgctctagggcaatcatggcatcctgagcattttgtctgtactcattgcaaagaagagattggctccagtcccttctttgagcggagtggcttggcctactgccccaacgactaccaccaacttttttctccacgctgtgcttactgcgctgctcccatcctggataaagtgctgacagcaatgaaccagacctggcacccagagcacttcttctgctctcactgcggagaggtgtttggtgcagaaggctttcatgagaaggacaagaagccatattgccgaaaggatttcttagccatgttctcacccaagtgtggtggctgcaatcgcccagtgttggaaaactacctttcagccatggacactgtctggcacccagagtgctttgtttgtggggactgcttcaccagtttttctactggctccttctttgaactggatggacgtccattctgtgagctccattaccatcaccgccggggaacgctctgccatgggtgtgggcagcccatcactggccgttgtatcagtgccatggggtacaagttccatcctgagcactttgtgtgtgctttctgcctgacacagttgtcgaagggcattttcagggagcagaatgacaagacctattgtcaaccttgcttcaataagctcttcccactgtaatgccaactgatccatagcctcttcagattccttataaaatttaaaccaagagaggagaggaaagggtaaattttctgttactgaccttctgcttaatagtcttatagaaaaaggaaaggtgatgagcaaataaaggaacttctagactttacatgactaggctgataatcttattttttaggcttctatacagttaattctataaattctctttctccctctcttctccaatcaagcacttggagttagatctaggtccttctatctcgtccctctacagatgtattttccacttgcataattcatgccaacactggttttcttaggtttctccattttcacctctagtgatggccctactcatatcttctctaatttggtcctgatacttgtttcttttcacgttttcccatttgccctgtggctcactgtcttacaatcactgctgtggaatcatgataccacttttagctctttgcatcttccttcagtgtatttttgtttttcaagaggaagtagattttaactggacaactttgagtactgacatcattgataaataaactggcttgtggtttcaataaaaaaaaaaaaaaaaaaaaaaaaaaaaaaaaaaaaaaa

SEQ ID NO: 2 - protein, 386 aa, Homo sapiens
MEELDALLEELERSTLQDSDEYSNPAPLPLDQHSRKETNLDETSEILSIQDNTSPLPAQLVYTTNIQELNVYSEAQEPKESPPPSKTSAAAQLDELMAHLTEMQAKVAVRADAGKKHLPDKQDHKASLDSMLGGLEQELQDLGIATVPKGHCASCQKPIAGKVIHALGQSWHPEHFVCTHCKEEIGSSPFFERSGLAYCPNDYHQLFSPRCAYCAAPILDKVLTAMNQTWHPEHFFCSHCGEVFGAEGFHEKDKKPYCRKDFLAMFSPKCGGCNRPVLENYLSAMDTVWHPECFVCGDCFTSFSTGSFFELDGRPFCELHYHHRRGTLCHGCGQPITGRCISAMGYKFHPEHFVCAFCLTQLSKGIFREQNDKTYCQPCFNKLFPL

SEQ ID NO: 3 - DNA, 389 bp, Homo sapiens
  Nucleotides 55 and 120 are either A, T, C, or G
ggtggaattcaggacactgagattgaaagggcctcctctcagtctccagtgtagntttcagggctcaacctgcaacagtgaattcctgattttatctctcctctcagtctccagtgtagntttcagggctcaacctgcaacagtgaattcctgattttatctctccaggctttcatgagaaggacaagaagccatattgccgaaaggatttcttagccatgttctcacccaagtgtggtggctgcaatcgcccagtgttggaaaactacctttcagccatggacactgtctggcacccagagtgctttgtttgtggggactgcttcaccagtttttctactggctccttctttgaactggatggacgtccattctgtgagctccattac

SEQ ID NO: 4 - DNA, 31 bp, Artificial Sequence
  Description of Artificial Sequence primer
atatctcgagaagagttagatgccttattgg

SEQ ID NO: 5 - DNA, 30 bp, Artificial Sequence
  Description of Artificial Sequence primer
atataagctttcagcccttgggcactgtgg

SEQ ID NO: 6 - DNA, 28 bp, Artificial Sequence
  Description of Artificial Sequence primer
atatctcgagccacagtgcccaagggcc

SEQ ID NO: 7 - DNA, 28 bp, Artificial Sequence
  Description of Artificial Sequence primer
atataagcttttacagtgggaagagctt

SEQ ID NO: 8 - DNA, 290 bp, Homo sapiens
attgaaaccacaagccagtttatttatcaatgatgtcagtactcaaagttgtccagttaaaatctacttcctcttgaaaaacaaaaatacactgaaggaagatgcaaagagctaaaagtggtatcatgattccacagcagtgattgtaagacagtgagccacaggggaaatgggaaaacgtgaaaagaaacaagttcaggaccaaattagagaagattgagtagggcatcactagaggtgaaaatggagaaacctaagaaaaccagtgttggcatgaattatgcaagtgg

SEQ ID NO: 9 - DNA, 454 bp, Mus musculus
gttctccccaaatgtggtggctgcaaccgcccagtgctggaaaactacctttcagccatgaacactgtctggcacccagagtgctttgtgtgtggggactgcttcagtagtttttcttctggctccttctttgaactggatggccgtcctttctgtgaactccattaccatcaccgccgagggaccctctgccatgactgtgggcagcccatcactggccgttgcatcagtgccatgggacataaatttcatcctgagcacttcgtgtgtgctttctgcctgacacagctgccgaaacgatcttcaaggagcagaacaacaagacctactgtgaaaaatgcttcactaagctcttttcacagtagttctcctttgactcacatctgcttcatgttgcctataaaactgaggccaagataggaaagagcatagattctgtccccagccttctgtt

SEQ ID NO: 10 - DNA, 424 bp, Mus musculus
gctgccgaaacagtcttcaaggagcagaacaacaagacctactgtgaaaaatgcttcactaagctcttttcacagtagttctcctttgactcacatctgcttcatgttgcctataaaactgaggccaagataggaaagagcatagattctgtccccagccttctgttcagtgggctaatggagtacaaacgggcctttgtagatgttactaggcatataccttcattttgaagatgcttctcattactttttaattatatatttagatttagatcatgctatcaagagcctctgaaggtatatttcttatgtgcacagttctttccactgctggttttctctttctcacctttgacctctgctgatgctccattcaaatcttcagatgtgggccacagggttttactctctctttgctgtcagg

SEQ ID NO: 11 - DNA, 494 bp, Mus musculus
gggagcagaacaacaagacctactgtgaaaaatgcttcactaagctcttttcacagtagttctcctttgactcacatctgcttcatgttgcctataaaactgaggccaagataggaaagagcatagattctgtccccagccttctgttcagtgggctaatggagtacaaacgggcctttgtagatgttactaggcatataccttcattttgaagatgcttctcattactttttaattatatatttagatttagatcatgctatcaagagcctctgaaggtatatttcttatgtgcacagttctttccactgctggttttctctttctcacctttgacctctgctgatgctccattcaaatcttcagatgtgggccacagggttttactctctctttgctgtcaggggtcacgcaatagtcttaaacccttagtgtctctgcattttgctttttaagcggaagtgtatttttactgacacatgtgatgaataaag

SEQ ID NO: 12 - DNA, 29 bp, Artificial Sequence
  Description of Artificial Sequence primer
atataagcttgccgaaacgatcttcaagg

SEQ ID NO: 13 - DNA, 29 bp, Artificial Sequence
  Description of Artificial Sequence primer
atatgtcgaccctgacagcaaagagagag

SEQ ID NO: 14 - DNA, 29 bp, Artificial Sequence
  Description of Artificial Sequence primer
atatgtcgaccagtgggaagagcttattg

SEQ ID NO: 15 - DNA, 31 bp, Artificial Sequence
  Description of Artificial Sequence primer
atatgcggccgcgatggaagagttagatgcc

SEQ ID NO: 16 - DNA, 27 bp, Artificial Sequence
  Description of Artificial Sequence primer
atagaattcgaagagttagatgcctta

SEQ ID NO: 17 - DNA, 27 bp, Artificial Sequence
  Description of Artificial Sequence primer
atactcgagcttgggcactgtggcaat

SEQ ID NO: 18 - DNA, 39 bp, Artificial Sequence
  Description of Artificial Sequence primer
tccccggaattctaagcggcagctgcttattggaggaac

SEQ ID NO: 19 - protein, 7 aa, Homo sapiens
EELDALL

SEQ ID NO: 20 - protein, 7 aa, Artificial Sequence
  Description of Artificial Sequence modified human sequence
EEAAALL

SEQ ID NO: 21 - DNA, 39 bp, Artificial Sequence
  Description of Artificial Sequence primer
aaggagactaaccttgctgcggcttcggagatcctttct

SEQ ID NO: 22 - protein, 6 aa, Homo sapiens
NLDETS

SEQ ID NO: 23 - protein, 6 aa, Artificial Sequence
  Description of Artificial Sequence modified human sequence
NLAAAS

SEQ ID NO: 24 - DNA, 39 bp, Artificial Sequence; identical to SEQ ID NO: 21
  Description of Artificial Sequence primer

SEQ ID NO: 25 - protein, 6 aa, Homo sapiens
QLDEML

SEQ ID NO: 26 - protein, 6 aa, Artificial Sequence
  Description of Artificial Sequence modified human sequence
QAAALM

SEQ ID NO: 27 - DNA, 39 bp, Artificial Sequence
  Description of Artificial Sequence primer
gatcacaaggcctccgcggccgcaatgcttgggggtctg

SEQ ID NO: 28 - protein, 5 aa, Homo sapiens
SLDSM

SEQ ID NO: 29 - protein, 5 aa, Artificial Sequence
  Description of Artificial Sequence modified human sequence
SAAAS

SEQ ID NO: 30 - DNA, 28 bp, Artificial Sequence
  Description of Artificial Sequence primer
atatgaattcgctcagttggatgagctc

SEQ ID NO: 31 - DNA, 29 bp, Artificial Sequence
  Description of Artificial Sequence primer
atatctcgagtcaagcatctgctcactgc

SEQ ID NO: 32 - protein, 15 aa, Homo sapiens
AQLDELMAHLTEMQA

SEQ ID NO: 33 - DNA, 33 bp, Artificial Sequence
  Description of Artificial Sequence primer
cccatatctcgagcaatggaagagttagatgcc

SEQ ID NO: 34 - DNA, 30 bp, Artificial Sequence
  Description of Artificial Sequence primer
cccataaagcttttacagtgggaagagctt

SEQ ID NO: 35 - DNA, 31 bp, Artificial Sequence; identical to SEQ ID NO: 4
  Description of Artificial Sequence primer

SEQ ID NO: 36 - DNA, 29 bp, Artificial Sequence
  Description of Artificial Sequence primer
atataagctttcagcccttgggcactgtg

SEQ ID NO: 37 - DNA, 25 bp, Artificial Sequence
  Description of Artificial Sequence primer
atatctcgaggccacagtgcccaag

SEQ ID NO: 38 - DNA, 28 bp, Artificial Sequence
  Description of Artificial Sequence primer
atataagcttttacagtgggaagagctt